(19) United States
(12) Patent Application Publication
(10) Pub. No.: US 2026/0097235 A1
(43) Pub. Date: Apr. 9, 2026

(54) PRE-TREATMENT PROCESSING OF PHASE-CONTRAST AND/OR DARK-FIELD IMAGE DATA FOR RADIATION THERAPY

(71) Applicant: SIEMENS HEALTHINEERS INTERNATIONAL AG, Steinhausen (CH)

(72) Inventors: Vidhya KRISHNAMURTHI, Los Altos, CA (US); Jörg FREUDENBERGER, Kalchreuth (DE); Thomas WEBER, Hausen (DE); Michael FOLKERTS, Costa Mesa, CA (US)

(73) Assignee: SIEMENS HEALTHINEERS INTERNATIONAL AG, Steinhausen (CH)

(21) Appl. No.: 18/906,177

(22) Filed: Oct. 4, 2024

(51) Int. Cl. A61N 5/10; G16H 20/40; G16H 30/40

(52) U.S. Cl. CPC A61N 5/1037; A61N 5/1045; G16H 20/40; G16H 30/40

(57) ABSTRACT

Example methods and systems for image data processing for radiation therapy are provided. In one example, a computer system may obtain planning image data that is generated using an imaging source to emit an imaging beam towards a patient and a detector to image a target structure within the patient during a pre-treatment phase of radiation therapy. Based on the planning image data, the computer system may generate at least one of (a) phase-contrast image data associated with the target structure and (b) dark-field image data associated with the target structure. The computer system may generate template image data associated with the target structure by processing at least one of the following: (a) the phase-contrast image data, (b) the dark-field image data and (c) derived image data that is generated based on the phase-contrast image data or the dark-field image data.

### CROSS-REFERENCE TO RELATED APPLICATION

The present application (Attorney Docket No. 124-0070-US2) is related in subject matter to U.S. patent application Ser. No. 18/809,498, filed on Aug. 20, 2024, which is incorporated herein by reference.

### BACKGROUND

Radiation therapy is a widely used cancer treatment modality that uses high-energy radiation to reduce or eliminate cancerous tumors. In practice, applied radiation does not inherently discriminate between a tumor and proximal healthy structures, such as organs, healthy tissues, etc. Ideally, the objective is to deliver a lethal or curative radiation dose to the tumor, while maintaining an acceptable dose level in the proximal healthy structures. During treatment time, delivery of planned radiation dose may be hindered by the presence of patient motion. In this case, motion management may be performed by tracking the position of a moving target structure and acting upon any deviation from a planned position. Conventionally, one approach for target structure tracking involves tracking fiducial markers/transponders that have been implanted into patients. However, the implantation of such markers/transponders may carry additional risks. In some cases, after implantation, some markers might migrate and become unreliable.

### BRIEF DESCRIPTION OF DRAWINGS

FIG. 1 is a schematic diagram illustrating an overview process for radiation therapy according to examples of the present disclosure;

FIG. 2 is a schematic diagram illustrating an example radiation therapy system that includes a grating-based imaging system capable of performing phase-contrast and/or dark-field imaging during a pre-treatment phase;

FIG. 3 is a schematic diagram illustrating a detailed example configuration of the grating-based imaging system shown in FIG. 1;

FIG. 4 is a schematic diagram illustrating an example process for a computer system to perform pre-treatment processing of phase-contrast and/or dark-field image data for radiation therapy;

FIG. 5 is a flowchart of an example detailed process for a computer system to generate absorption image data, phase-contrast image data and dark-field image data;

FIG. 6 is a diagram illustrating example phase-stepping curves that are generated based on planning image data and reference image data;

FIG. 7 is a flowchart illustrating an example detailed process for processing image data and generating derived image data during a pre-treatment phase of radiation therapy;

FIG. 8A is a schematic diagram illustrating a first example for template generation based on phase-contrast and/or dark-field image data;

FIG. 8B is a schematic diagram illustrating a second example for template generation based on phase-contrast and/or dark-field image data using an artificial intelligence (AI) engine;

FIG. 9 is a schematic diagram illustrating an example process for a computer system to perform target structure tracking based on template image data; and

FIG. 10 is an example radiation therapy system that includes a grating-based imaging system capable of performing phase-contrast and/or dark-field imaging during a treatment phase.

### SUMMARY

According to examples of the present disclosure, phase-contrast and/or dark-field imaging may be implemented during a pre-treatment phase of radiation therapy to facilitate, inter alia, template generation for target structure tracking. As used herein, the term “target structure” may refer generally to any suitable structure that requires tracking, such as tumor, organ-at-risk (OAR), healthy tissue, bony structure (e.g., vertebra), implanted marker, brachytherapy applicator for brachytherapy, etc. The term “template image data” or “template image” may refer generally to reference image data that is generated during a pre-treatment phase of radiation therapy for tracking target structure(s) during a subsequent treatment phase. The term “template generation” may refer generally to a process for generating template image data. The term “target structure tracking” may refer generally to estimating position data associated with a target structure, such as to facilitate motion management, position monitoring and/or verification, target localization or the like during radiation treatment.

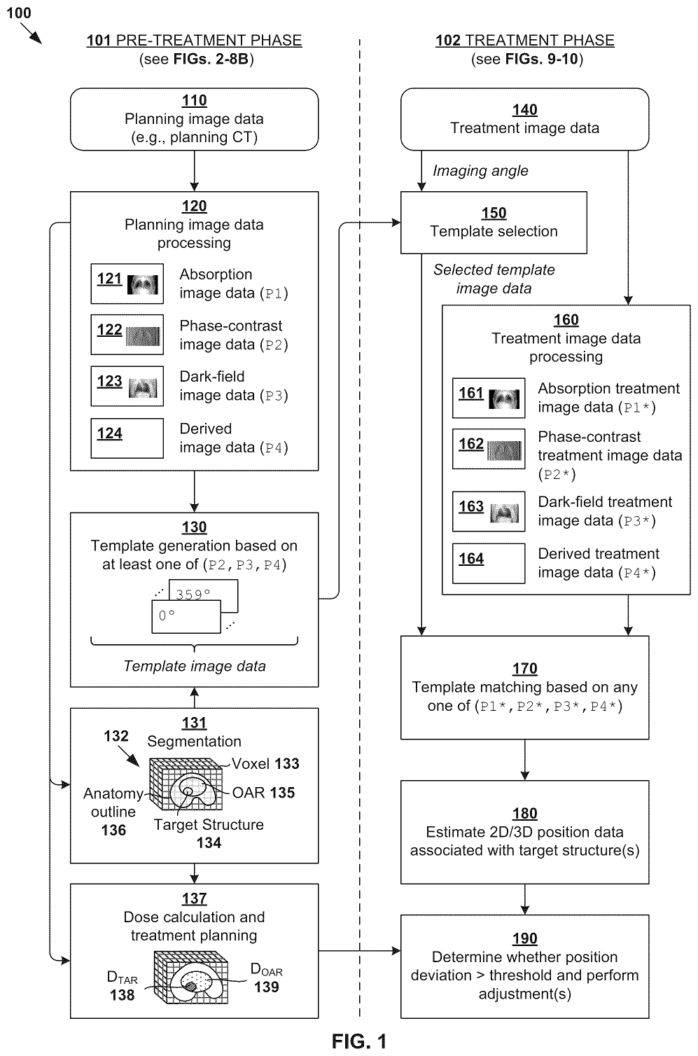

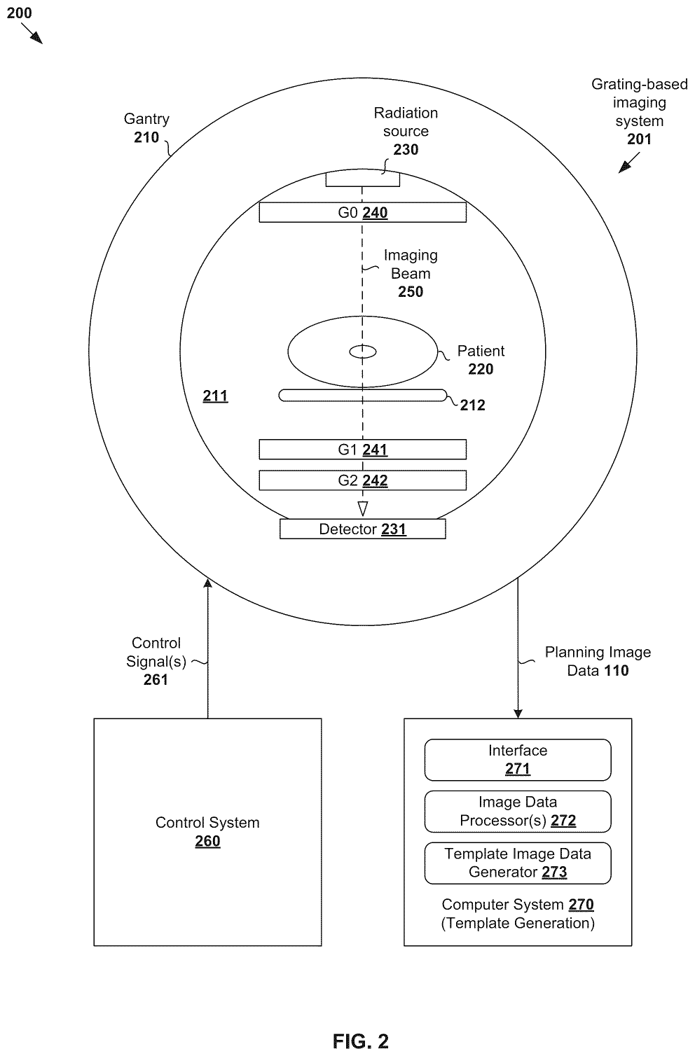

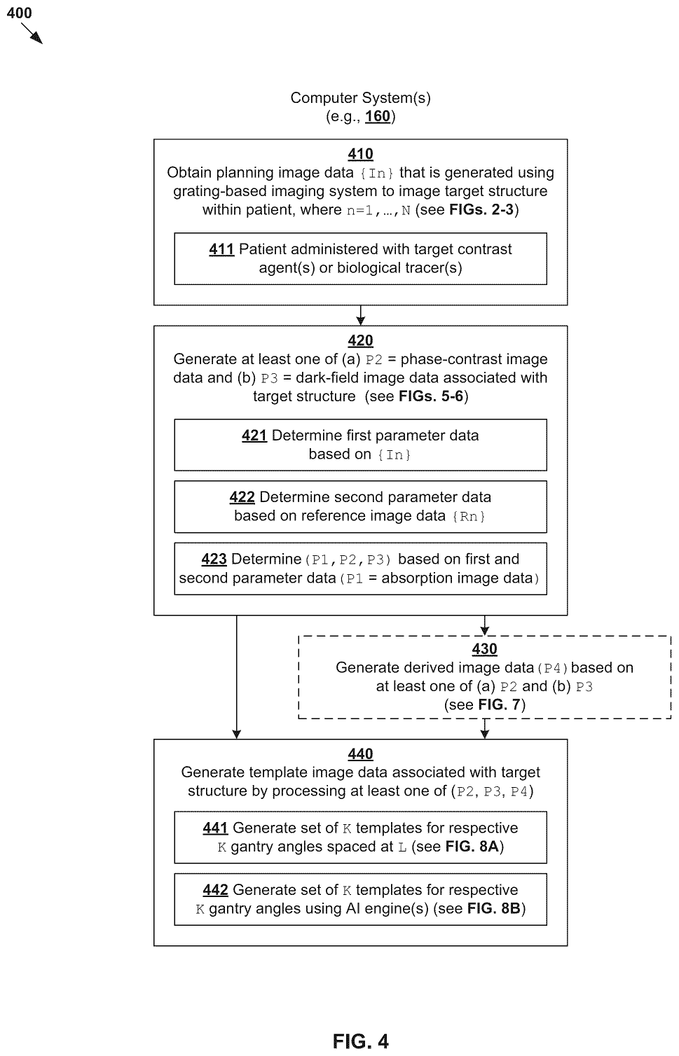

According to a first aspect, examples of the present disclosure provide method(s) and computer system(s) for pre-treatment processing of phase-contrast and/or dark-field image data for radiation therapy. In one example, a computer system (see 270 in FIG. 2) may obtain planning image data (see 110 in FIG. 1) that is generated using an imaging source to emit an imaging beam towards a patient and a detector to image a target structure within the patient during a pre-treatment phase of radiation therapy. Based on the planning image data, the computer system may generate at least one of (a) phase-contrast image data associated with the target structure and (b) dark-field image data associated with the target structure. The computer system may generate template image data associated with the target structure by processing at least one of the following: (a) the phase-contrast image data, (b) the dark-field image data and (c) derived image data that is generated based on the phase-contrast image data or the dark-field image data. The template image data may be used for tracking the target structure during a treatment phase of radiation therapy. See also 110-130 in FIG. 1 and 410-440 in FIG. 4.

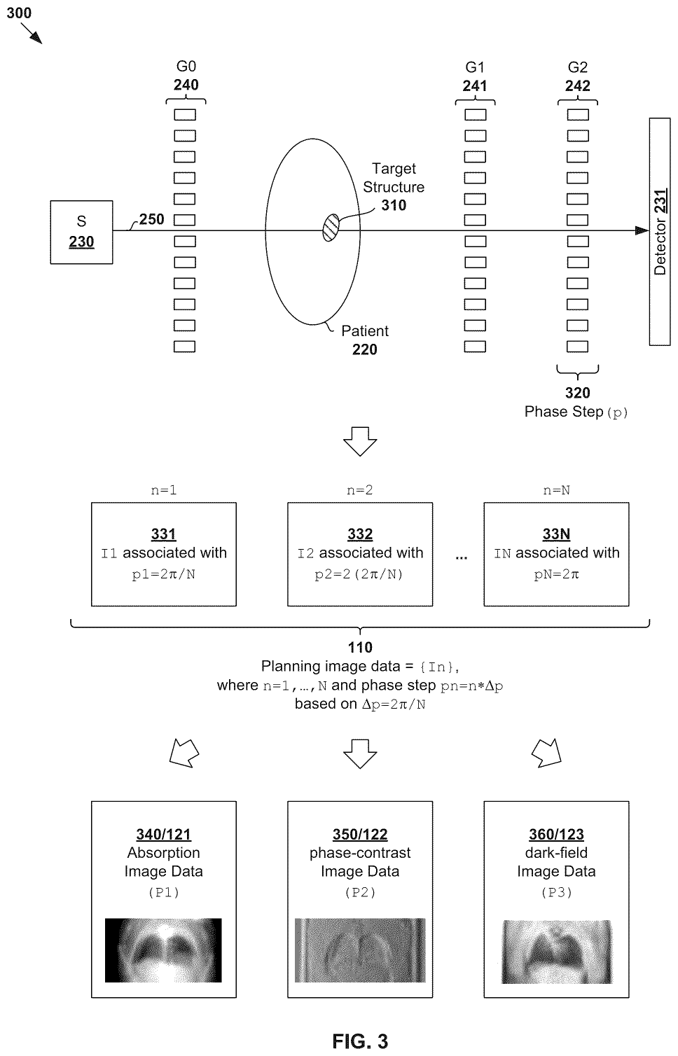

According to a second aspect, examples of the present disclosure provide radiation therapy system(s) for pre-treatment processing of phase-contrast and/or dark-field image data for radiation therapy. In one example, a radiation therapy system (see 200 in FIG. 2) may include a grating-based imaging system and a computer system for template generation. The grating-based imaging system (see 240 in FIGS. 2-3) may include an imaging source, a detector and multiple gratings that are positioned between the imaging source and the detector. The computer system (see 270 in FIG. 2) may obtain, from the grating-based imaging system, planning image data that is generated using the imaging source to emit an imaging beam towards the multiple gratings and the detector to image a target structure within the patient during a pre-treatment phase of radiation therapy. Based on the planning image data, the computer system may generate at least one of (a) phase-contrast image data associated with the target structure and (b) dark-field image data associated with the target structure. The computer system may generate template image data associated with the target structure by processing at least one of the following: (a) the phase-contrast image data, (b) the dark-field image data and (c) derived image data that is generated based on the phase-contrast image data or the dark-field image data. The template image data may be used for tracking the target structure during the treatment phase of the radiation therapy. See also 110-130 in FIG. 1 and 410-440 in FIG. 4.

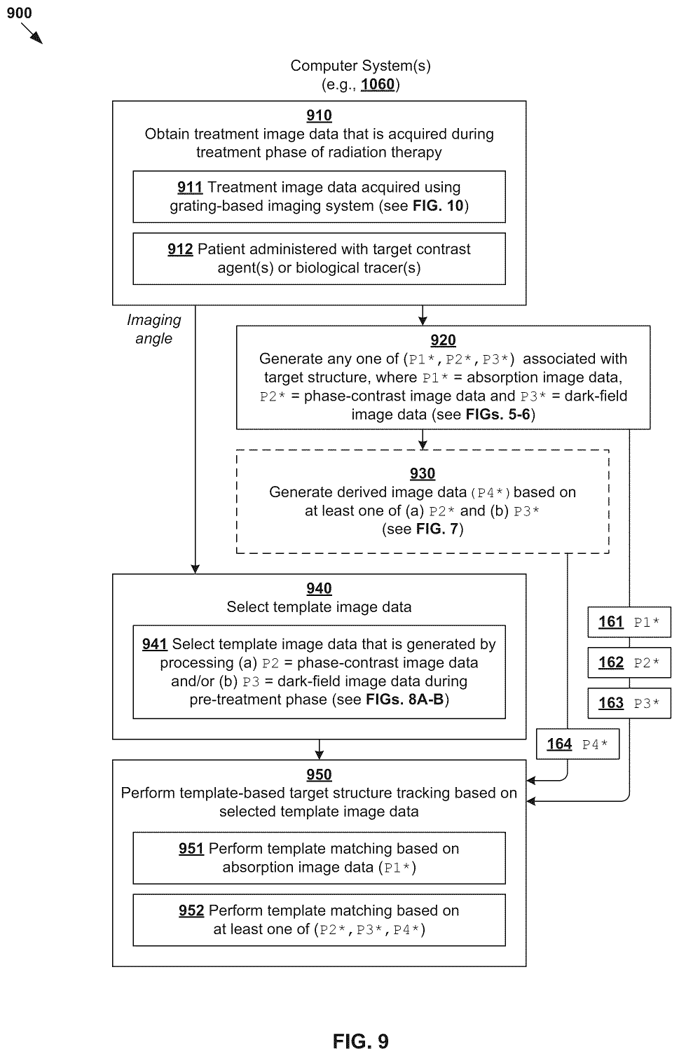

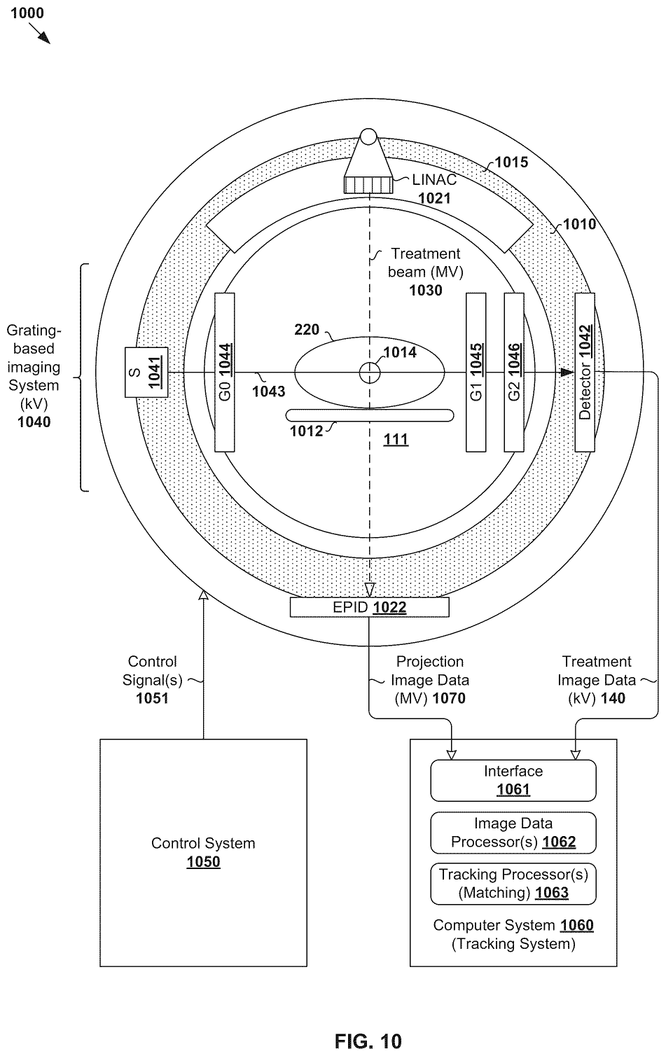

According to a third aspect, examples of the present disclosure provide method(s) and computer system(s) for target structure tracking. In one example, a computer system (e.g., 1060 in FIG. 10) may obtain treatment image data that is generated using an imaging source to emit an imaging beam towards a patient and a detector to image a target structure within the patient during a treatment phase of radiation therapy. The computer system may select template image data and determine position data associated with the target structure based on the selected template image data and the treatment image data. The selected template image data may be generated based on at least one of the following: (a) phase-contrast image data associated with the target structure, (b) dark-field image data associated with the target structure and (c) derived image data that is generated based on the phase-contrast image data or the dark-field image data. See 140-150 and 170-190 in FIG. 1, and FIG. 910-950 in FIG. 9.

Examples of the present disclosure may further include a non-transitory computer-readable storage medium that includes a set of instructions which, in response to execution by a processor, cause the processor to perform aspect(s) of the above method(s) according to the first aspect or the second aspect. A further aspect may include a radiation therapy system that includes a grating-based imaging system and computer system to perform target structure tracking according to the third aspect.

Using examples of the present disclosure, phase-contrast image data and/or dark-field image data may be generated to provide additional information associated with a target structure compared to absorption image data. The additional information may be used during a pre-treatment phase of radiation therapy to generate template image data with improved quality, such as improved soft tissue contrast and/or target structure visibility for use in target structure tracking. Using examples of the present disclosure, target margins may be reduced to enhance the sparing of healthy tissue surrounding target structure(s) during treatment. In practice, examples of the present disclosure may be implemented to improve motion management, dose accuracy and conformity and sparing of healthy tissue during a treatment phase of radiation therapy. Examples of the present disclosure should be contrasted against conventional approaches that rely on conventional X-ray imaging that only generates absorption image data for template generation and target structure tracking.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated herein.

Although the terms “first” and “second” are used to describe various elements, these elements should not be limited by these terms. These terms are used to distinguish one element from another. For example, a first element may be referred to as a second element, and vice versa. As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items. By way of example, the phrases “at least one of A, B, and C” or “at least one of A, B, or C” each refer to only A, only B, or only C; and/or any combination of A, B, and C. In instances where it is intended that a selection be of “at least one of each of A, B, and C,” or alternatively, “at least one of A, at least one of B, and at least one of C,”it is expressly described as such.

### Overview

FIG. 1 is a flow diagram illustrating example overview process 100 for radiation therapy. Example process 100 may include one or more operations, functions, or actions illustrated by one or more blocks. The various blocks may be combined into fewer blocks, divided into additional blocks, and/or eliminated based upon the desired implementation. In the example in FIG. 1, radiation therapy may include (a) a pre-treatment phase (see 101) to generate a treatment plan (see 137) for a patient requiring radiation therapy and (b) a treatment phase (see 102) to deliver treatment according to the treatment plan. The goal of the treatment plan is to deliver high radiation dose to a target structure (e.g., lung tumor) and lower radiation dose to proximal organs-at-risk (OARs) and healthy tissues (e.g., central airway).

One important factor for effective treatment delivery is the location of the target structure within a planning target volume (PTV) to which high radiation dose is delivered. In practice, the patient or the tumor may move outside of the PTV due to infrafraction motion (e.g., respiratory motion, patient's movement, etc.). One way of managing motion is tracking the target structure using a template-based, markerless approach that does not rely on invasive metal markers/transponders implanted into the patient. In the example in FIG. 1, markerless target structure tracking may involve (a) template generation (see 130) during pre-treatment phase 101 and (b) template matching (see 170) during treatment phase 102. This way, any deviation from a planned position may be detected during treatment delivery.

### (a) Pre-Treatment Phase

In more detail, at 110 in FIG. 1, planning image data may be acquired during pre-treatment phase 101 using any suitable imaging modality. In one example, planning image data 110 may be planning computed tomography (CT) data that is acquired using a CT imaging system. In another example, planning image data 110 may be cone-based computed tomography (CBCT) data that is acquired using a CBCT imaging system. Pre-treatment phase 101 is also known as a treatment planning phase. As used herein, the term “planning image data” may refer generally to image data that is acquired during a pre-treatment or planning phase of radiation therapy.

At 120 in FIG. 1, planning image data 110 may be processed to generate absorption image data (P1) 121, phase-contrast image data (P2) 122 and/or dark-field image data (P3) 123. Additionally, derived image data (P4) 124 may be generated based on P2 122 and/or P3 123. Image data 121-124 will be explained using FIGS. 2-7.

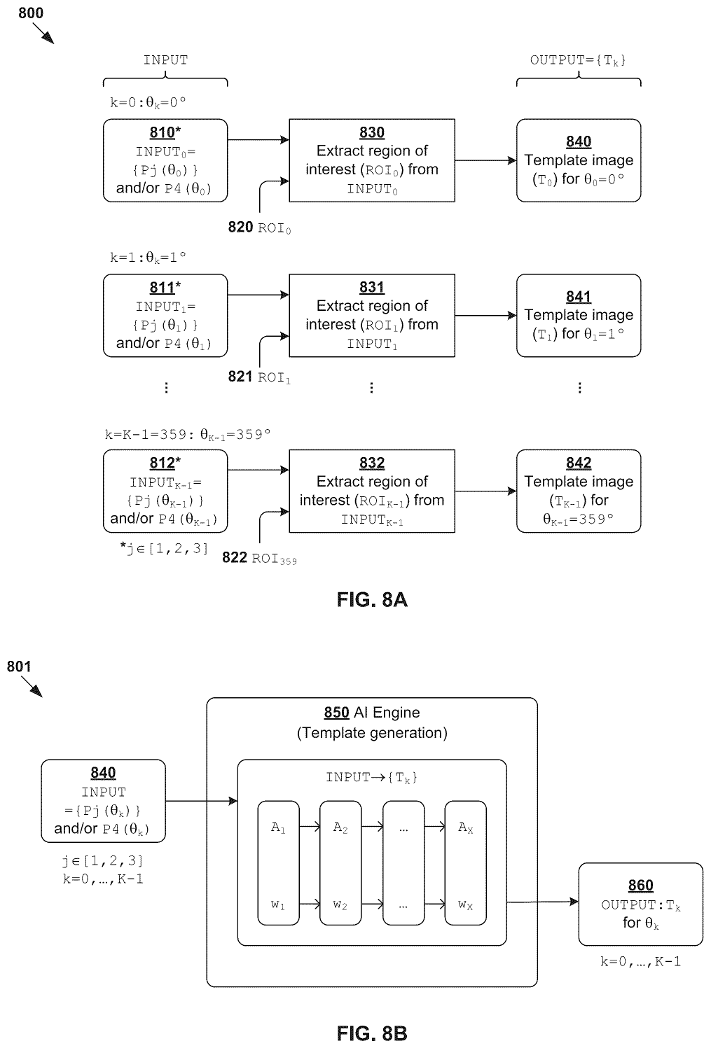

At 130 in FIG. 1, template generation may be performed to generate template image data by processing at least one of P2 122, P3 123 and P4 124. For example, in order to be able to track from all gantry angles, the template image data may include a set of K template images for K=360 gantry angles that are spaced at L=1 degree. Some examples will be explained using FIGS. 8A-B. Any additional data may be used for template generation, such as segmentation data relating to contoured surfaces, etc.

Depending on the desired implementation, segmentation (see 131 in FIG. 1) may be performed based on one or more of P1 121, P2 122, P3 123 and P4 124. In practice, segmentation may be performed to generate three-dimensional (3D) volume image data 132 identifying the contour, shape, size, and location of patient's anatomy 2126, target structure 134 (e.g., tumor), OAR 135, or any other structure of interest (e.g., soft tissue, bone). Volume image data 132 (also known as a digital or treatment volume) may be divided into multiple smaller volume-pixels (voxels) 133, each representing a 3D element within the treatment volume. Segmentation may be performed manually (e.g., drawn by a physician) or using any suitable software (e.g., segmentation software/AI engine(s)).

In practice, volume image data 132 may include multiple target structures and irregularly shaped voxels. Although shown as having a regular shape (e.g., cube), voxel 133 may have any suitable shape (e.g., non-regular). Any additional and/or alternative data may be used, such as prescription data, disease staging data, biologic or radiomic data, genetic data, assay data, biopsy data, past treatment or medical history, any combination thereof, etc. Further, at 137 in FIG. 1, pre-treatment phase 101 may include dose calculation to generate dose data specifying radiation doses to be delivered to target structure 134 (denoted “D_{TAR}” at 138) and OAR 135 (denoted “D_{OAR}” at 139). For example, target structure 134 may represent a malignant tumor requiring radiotherapy treatment, such as lung tumor, prostate tumor, etc. OAR 135 may be a proximal healthy structure that might be adversely affected by the treatment, such as central airway, rectum, bladder, etc.

Treatment planning may be performed based on segmentation data 131, dose data 138-139 and/or any of image data 121-124. In practice, a treatment plan (not shown in FIG. 1) may be generated to include two-dimensional (2D) fluence map data for a set of beam orientations or angles. Each fluence map may specify the intensity and shape (e.g., as determined by an MLC) of a radiation beam emitted from a radiation source at a particular beam orientation and at a particular time. For example, intensity modulated radiotherapy treatment (IMRT) or any other treatment technique(s) may involve varying the shape and intensity of the radiation beam while at a constant gantry and couch angle. Alternatively or additionally, the treatment plan may include machine control point data (e.g., jaw and leaf positions), volumetric modulated arc therapy (VMAT) trajectory data for controlling a treatment delivery system, etc. In practice, the treatment plan may be generated based on goal doses prescribed by a clinician (e.g., oncologist, dosimetrist, planner, etc.), such as based on the clinician's experience, the type and extent of the tumor, patient geometry and condition, etc.

### (b) Treatment Phase

At 140 in FIG. 1, treatment image data associated with target structure(s) 134 of the patient may be continuously acquired during irradiation, such as using a treatment delivery machine that includes an imaging system to facilitate target structure tracking. At 150, template selection may include selecting template image data, such as template image(s) associated with a gantry angle that is closest to an imaging angle of treatment image data 140, etc. As used herein, the term “treatment image data” may refer generally to image data that is acquired during a treatment phase of radiation therapy.

At 160 in FIG. 1, any suitable image data processing may be performed to improve the quality of treatment image data 140 prior to subsequent template matching. For example, when grating-based imaging is used, treatment image data 140 may be processed to generate absorption treatment image data (P1*) 161, phase-contrast treatment image data (P2*) 162 and/or dark-field treatment image data (P3*) 163.

Further, derived treatment image data (P4*) 164 may be generated based on P2* 162 and/or P3* 163.

At 170 in FIG. 1, during template matching, any suitable approach may be implemented to identify a match (e.g., best match) between the selected template image data and treatment image data 140. In one example, template matching may be performed based on P1* 161. In another example, template matching may be performed based on at least one of the following: P2* 162, P2* 163 and P4* 164. As will be described below using FIGS. 9-10, template matching approach may involve calculating normalized cross-correlation data associated with all possible template locations within a specified search region as a measure of similarity.

At 180 in FIG. 1, the resulting match of template matching 170 may include the current 2D position data of target structure 134. Additionally, 3D position data may be estimated by performing triangulation based on the current 2D position data as well as the 2D position data associated with previous gantry angles. At 190, any adjustment may be performed in response to detecting an excessive positional displacement (e.g., threshold exceeded), such as to interrupt the treatment and/or recommend a patient repositioning, etc.

Using examples of the present disclosure, template image data that is generated based on phase-contrast and/or dark-filed image data during pre-treatment phase 101 may be used for comparison with treatment image data 140 during treatment phase 102, such as to compensate for motion, evaluate the treatment response using 2D projection image data acquired during treatment, etc. For example, in relation to motion tracking, planning image data 110 acquired using CBCT scan may be used as a reference for motion tracking during treatment delivery. In relation to treatment response, the projected size of a tumor may be evaluated by comparing pre-treatment image data 122/123/124 (i.e., generated based on planning image data 110) and image data 162/163/164 (i.e., generated based on treatment image data 140). This is especially beneficial when the contrast in absorption image data is minimal or nonexistent compared to phase-contrast/dark-field/derived image data. This may also facilitate a potential reduction in radiation dose during treatment.

### Radiation Therapy System

FIG. 2 is a schematic diagram illustrating example radiation therapy system 200 that includes grating-based imaging system 210 capable of performing phase-contrast and/or dark-field imaging during pre-treatment phase 101. It should be understood that, depending on the desired implementation, example system 200 may include additional and/or alternative components than that shown in FIG. 2. Here, first example system 200 may include grating-based imaging system 210 to acquire planning image data 110, control system 260 to control operations of imaging system 210 and first computer system 270 to process planning image data 110 according to examples of the present disclosure.

In the example in FIG. 2 example system 200 may include gantry 210 having opening 211 and patient support 212 for supporting patient 220 requiring radiation therapy. In the example in FIG. 2, gantry 210 has a ring-based configuration. In an alternative example, gantry may have a C-arm configuration. Imaging system 210 may implement any suitable imaging modality for image data acquisition, such CT, CBCT, positron emission tomography (PET), single photon emission computed tomography (SPECT), magnetic resonance imaging (MRI), magnetic resonance tomography (MRT), any combination thereof, etc. For example, when CT is used, planning image data 110 (e.g., planning CT scan) may include a series of 2D projection images or slices (e.g., CT slices), each representing a cross-sectional view of the patient's anatomy. In practice, spectral CT data (e.g., dual energy CT (DECT) and photon counting CT) may be acquired to provide access to various quantities at the planning stage. When CBCT is used, planning image data 110 may include 2D projection images.

Radiation therapy system 200 may further include grating-based imaging system 201 to facilitate template generation, segmentation and treatment planning based on phase-contrast and/or dark-field image data. Grating-based imaging system 201 may include radiation source 230 (e.g., X-ray source) to project imaging beams 250 towards detector 231, which includes pixel detectors that are disposed opposite of radiation source 230. Control system 260 may be electrically coupled to gantry 210 to control operation(s) of gantry 210 using control signal(s) 261. Radiation source 230 may be configured to generate any suitable beam, such as fan beam, etc.

During an imaging procedure, gantry 210 may be rotated about opening 211 while radiation source 230 generates and directs X-ray beam(s) 250 along a projection line towards patient 220 and detector 231. Detector 231 may measure the X-ray absorption and produce a voltage proportional to the intensity of incident X-rays. The voltage may be read and digitized to generate planning image data 110. In practice, planning image data 110 may include image data acquired at different gantry angles. Although one pair of imaging source 230 and detector 231 is shown in FIG. 2, imaging system 210 may include multiple sources and/or detectors, such as to facilitate stereoscopic imaging, etc.

Example system 200 may further include first computer system 270 that is communicatively coupled with grating-based imaging system 201 to obtain and process planning image data 110 for template generation according to blocks 2110-130 in FIG. 1. In the example in FIG. 2, first computer system 270 may include interface 271 to interact with grating-based imaging system 201 to obtain planning image data 110; image data processor(s) 272 to process planning image data 110 to generate image data 121-124; and template generator 273 to generate template(s) based on at least one of image data 122-124. First computer system 270 may include any alternative and/or additional components not shown in FIG. 2. In practice, first computer system 270 may be implemented using a physical machine (bare metal machine) and/or virtual machine that is deployed in a cloud-based environment (i.e., not located in the same physical location as grating-based imaging system 201).

In practice, computer system 270 may be located in the same physical location as radiation therapy system 200, or in a different location. In both cases, computer system 270 may be communicatively coupled with radiation therapy system 200 via any suitable communication network(s). Computer system 270 may be implemented using one or more physical machines (bare metal machines) and/or virtual machines deployed in a cloud-based environment. Control system 260 and computer system 270 may include any display device(s) and user input device(s), which are not shown for simplicity.

### Phase-Contrast and/or Dark-Field Imaging

According to examples of the present disclosure, grating-based imaging system 201 may be configured to facilitate phase-contrast and/or dark-field imaging. A more detailed view is shown in FIG. 3, which is a schematic diagram illustrating detailed example configuration 300 of grating-based imaging system 201 shown in FIG. 2. In practice, X-ray phase-contrast and dark-field imaging may be performed with low-brilliance medical X-ray sources using Talbot-Lau interferometry. A description of Talbot-Lau interferometry may be found in F. Pfeiffer, T. Weitkamp, O. Bunk, and C. David, “Phase retrieval and differential phase-contrast imaging with low brilliance x-ray sources,”Nature Phys. 2, 258-261 (2006), which is incorporated herein by reference.

In the example in FIGS. 2-3, grating-based imaging system 201 may include multiple gratings (labelled “G0” to “G2”) that are interposed between source 230 and detector 231. In the case of three gratings, for example, first grating=source grating (G0) 240, second grating=phase grating (G1) 241 and third grating=analyzer grating (G2) 242 may be positioned between source 230 and detector 231. Depending on the exact properties of source 230 and detector 231, gratings 240-242 may have periods in the order of 2 to 50 micrometers (i.e., micrometer-scale gratings). The movement of source 230, detector 231 and multiple gratings 240-242 may be controlled using control system 260.

As used herein, the term “grating” may refer generally to an optical component or structure that includes a number of (e.g., evenly spaced) parallel lines or slits. These parallel lines or slits may diffract X-rays or light, creating interference patterns that may be used to enhance image contrast, such as for materials that are weakly absorbing and would otherwise show low contrast in traditional absorption-based imaging. The term “grating-based imaging system” may refer generally to an imaging system that is capable of performing phase-contrast and/or dark-field imaging, and includes at least an imaging source, a detector and multiple gratings. In practice, any suitable number of gratings (e.g., at least two) may be configured. Depending on the desired implementation, grating-based imaging system 201 may include a variety of X-ray energies (e.g., single energy or dual energy) and/or gratings to maximize or improve target visibility.

For example, G0 240 may be positioned downstream of the direction of wave propagation from source 230 to ensure spatial coherence by introducing multiple virtual slit sources. Wavefronts originating from the slit sources of G0 240 may impinge on target structure(s) 310 within patient 220, who is positioned between the G0 240 and G1 241. The wavefronts may be deformed by patient 220 depending on their material properties. Further towards detector 231, G1 241 may be deployed as a phase mask to imprint a periodic phase shift on the wavefronts emitted from G0 240. The resulting intensity pattern from G1 241 may be sampled by a measurement of intensity for a number of grating positions (p) of G2 242. Each grating position p is known as a phase step. The process of adjusting or shifting the phase step may be referred to as phase stepping. Depending on the desired implementation, phase stepping may be performed using active or passive methods. See 320 in FIG. 3.

In the example in FIG. 3, planning image data 110 acquired using grating-based imaging system 201 may include a set of multiple (N) planning images denoted as |¿| where n=1, . . . , N. Here, |¿| may be acquired sequentially using detector 231 with patient 220 interposed between G0 240 and G1 241. Note that the measurements may also be done continuously or passively by exploiting intrinsic vibrations of the setup or system. After each planning image (¿) is acquired, the grating position (p) of G2 242 may be moved by, for example, 2π/N of one period. In this case, the n^{th} planning image may be associated with a particular grating position pn, where n=1, . . . , N. For example, p1=¿2π/N for n=1, p2=¿2(2π/N) for n=2, and so on until pN=¿N(2π/N)=¿2π for n=N. See 331-33N in FIG. 3.

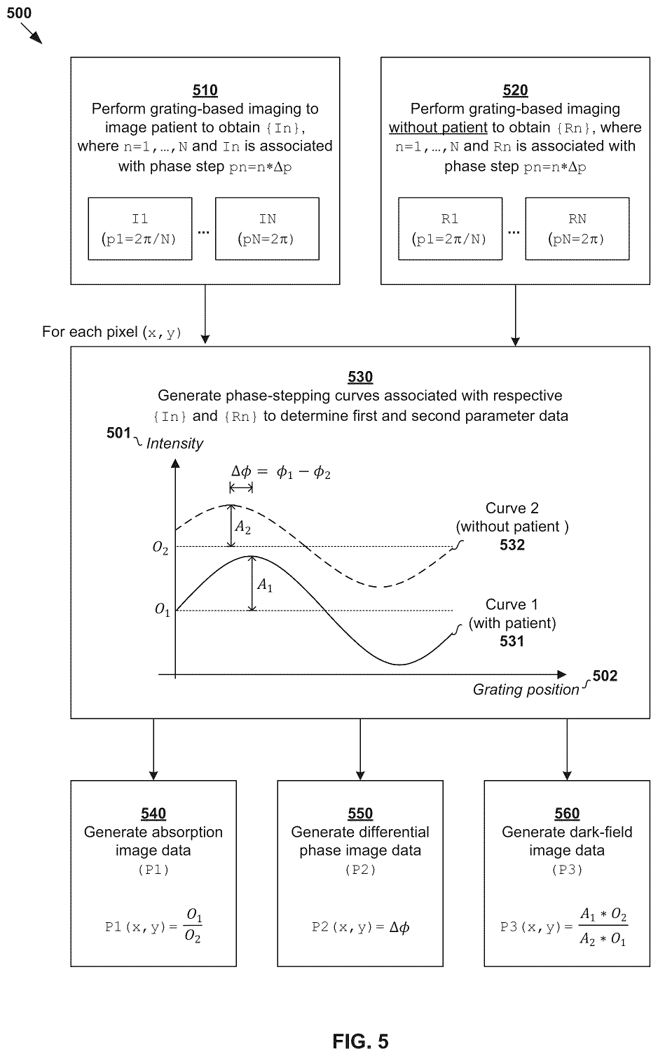

Based on planning image data 110, multiple types of image data may be generated or extracted to provide complementary contrasts, such as absorption image data 340/121 (denoted as P1), phase-contrast image data 250/122 (denoted as P2) and dark-field image data 360/123 (denoted as P3). Detailed examples for generating P1 210, P2 350 and P3 360 will be explained using FIGS. 4-5. As used herein, the term “absorption image data” (also known as “transmission image data”) may refer generally to image data that is generated based on attenuation of imaging beam(s). In general, absorption-based X-ray imaging may rely on the differential absorption of X-rays by different materials.

The term “phase-contrast image data” (also known as “differential phase image data”) may refer generally to image data that is generated based on a refraction property of imaging beam(s), such as phase shift(s) caused by the refraction. For example, when an X-ray wave passes through a particular material, it may bend slightly due to its interactions with the material's electron. This bending, which is called refraction, causes a shift in the phase of the X-ray wave. The phase shift may be detected to generate phase-contrast image data, which provides enhanced contrast information. This should be contrasted against standard X-ray imaging, which relies on how much the X-ray intensity varies as it passes through the material.

The term “dark-field image data” may refer generally to image data that is generated based on a scattering property of imaging beam(s). For example, a small-angle or ultra-small-angle scattering signal may be more sensitive to structural variations and/or density variations. Denser materials generally absorb more X-rays, leading to darker areas on the resulting image data. In practice, dark-field image data may provide a better visualization of fine structural details that may not be visible in absorption image data, thereby improving target visibility.

### Pre-Treatment Processing

According to examples of the present disclosure, template generation and treatment planning may be performed in an improved manner during pre-treatment phase 101. For example, template image data with improved target visibility and soft tissue contrast may be generated based on phase-contrast and/or dark-field image data. In practice, template generation may be performed to facilitate template-based, markerless target structure tracking (see 140-190 in FIG. 1). Any improvement in the accuracy of target structure tracking may in turn reduce the probability of target miss and/or the probability of healthy tissue damage during treatment delivery. Examples of the present disclosure may be implemented as part of any suitable software suite for template generation and/or treatment planning during pre-treatment phase 101, as well as target structure tracking during treatment phase 102 of radiation therapy.

In more detail, FIG. 4 is a flowchart illustrating example process 400 for computer system 270 to perform pre-treatment processing of phase-contrast and/or dark-field image data for radiation therapy. Example process 400 may include one or more operations, functions, or actions illustrated by one or more blocks, such as 410 to 440. Depending on the desired implementation, various blocks may be combined into fewer blocks, divided into additional blocks, and/or eliminated. Using the example in FIG. 1, example process 400 be performed using computer system 270 capable of acting as a template generation system. Computer system 270 may include any suitable module(s) or component(s) such as interface 271 to interface with radiation therapy system 200 to perform block 410, image data processor(s) 272 to perform blocks 420-430, template image data generator 273 to perform block 440, etc.

At 410 in FIG. 4, computer system 270 may obtain planning image data 110, which may be generated or acquired using grating-based imaging system 201 (i.e., imaging system equipped with a grating interferometer) that includes imaging source 230, detector 231 and multiple gratings 240-242 interposed between them 230-231.

Imaging source 230 may be configured to emit imaging beam(s) 250 towards gratings 240-242 and detector 231 to image target structure(s) 310 within patient 220 during pre-treatment phase 101.

At 420 in FIG. 4, based on planning image data 110, computer system 270 may generate at least one of (a) phase-contrast image data (P2) 350 associated with target structure 310 and (b) dark-field image data (P3) 360 associated with target structure 310. Depending on the desired implementation, block 420 may be performed based on planning image data 110 and reference image data, which is generated using grating-based imaging system 201 without patient 220 (i.e., no subject).

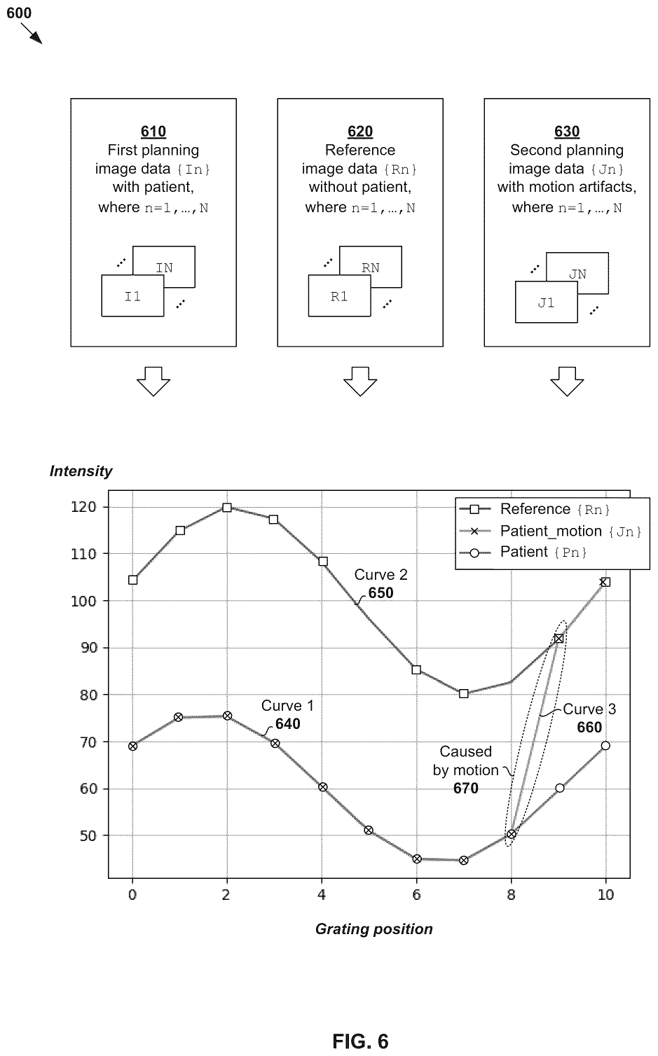

As will be discussed using FIGS. 5-6, block 420 may include determining first parameter data associated with planning image data 110 (see 421) and determining second parameter data associated with the reference image data (see 422). Based on the first parameter data and the second parameter data, various types of image data may be generated (see 423), such as P1 340, P2 350, P3 360 or any combination thereof. The first and second parameter data may be extracted from phase-stepping curves and include intensity offset data, intensity amplitude data, differential phase data (also known as phase shift data), visibility data, etc.

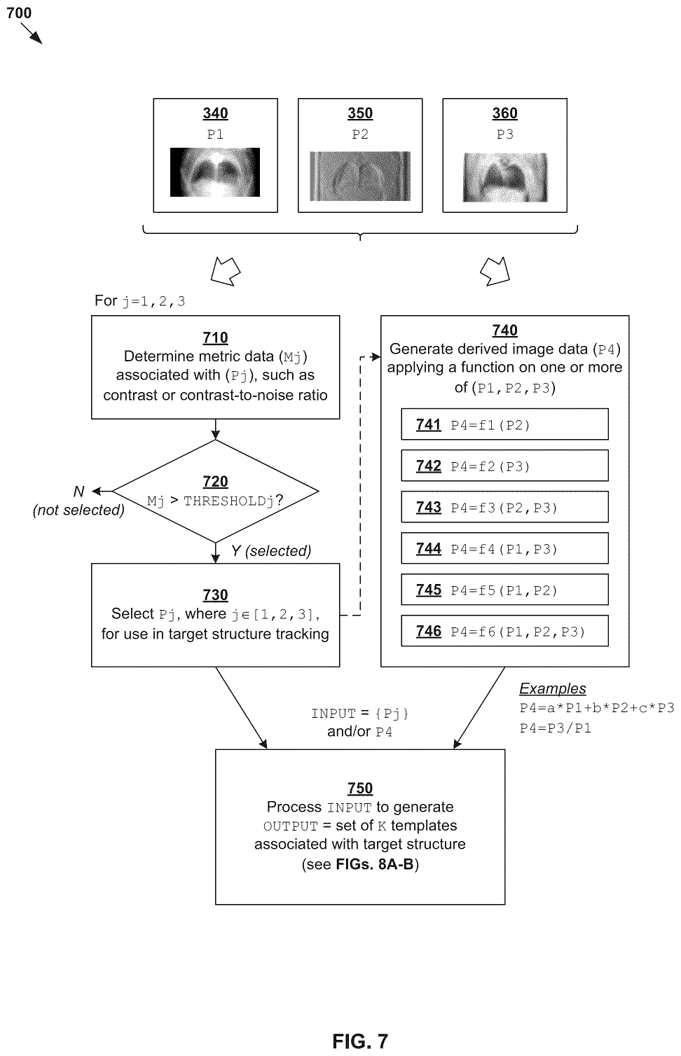

At 430 in FIG. 4, computer system 270 may generate derived image data (denoted as P4) based on at least one of (a) P2 350 and (b) P3 360 associated with target structure 310. Depending on the desired implementation, block 430 may involve applying any suitable function(s) to combine or calculate a ratio between at least two P1 340, P2 350 and P3 360. Some examples will be discussed below using FIG. 7.

At 440 in FIG. 4, computer system 270 may generate template image data associated with target structure 310 by processing at least one of the following: (a) P2 350, (b) P3 360 and (c) derived image data (P4). Using a template-based approach, the template image data may be used for tracking target structure 310 during treatment phase 102. Some examples for template generation will be discussed using FIGS. 8A-B.

Examples of the present disclosure may be implemented to take advantage of additional data provided by P2 350 and/or P3 360. In particular, P2 350 and P3 360 may employ fundamentally different physical properties of target structure 310, such as phase shift (i.e., real part of the refractive index) and small angle scattering that depend on a porosity characteristic of target structure 310. In the case of lung cancer treatment, lung tumors are known to be solid compared to surrounding lung tissue that is porous due to the alveoli. This usually results in a large signal difference between a tumor and healthy tissue in P3 360, where border(s) of the tumor may be more easily located during target structure tracking. Further, P2 350 is generally differential in nature in a grating-based phase-contrast imaging setup and expected to have strong signals at the border(s) of a solid structure.

Depending on the desired implementation, patient 220 may be administered with targeted contrast agents or biological tracers to enhance target visibility and improve the detectability of specific cells or cell clusters (see 411 in FIG. 4). Example biological tracers include microbubbles (i.e., ultrasound contrast agents), nanoparticles, peptides, etc. Such biological tracers may selectively bind to any specific cancer cells or healthy tissues to increase phase-contrast and/or dark-field properties. For example, microbubbles may each include a gas core that is surrounded by a surfactant or polymer shell, whose surface may be functionalized with a range of targets. Gold nanoparticles or microparticles may be loaded on tracers to create a more porous structure in or around a target tissue. Any suitable approach may be used to administer the biological tracers, such as intravenous injection, direct injection, oral ingestion, etc.

### Example Planning Image Data Processing

Blocks 410-420 will be explained further using FIG. 5, which is a flowchart illustrating example process 500 for computer system 270 to generate absorption image data 340, phase-contrast image data 350 and dark-field image data 360. Example process 500 may include one or more operations, functions, or actions illustrated by one or more blocks, such as 510 to 560. Depending on the desired implementation, various blocks may be combined into fewer blocks, divided into additional blocks, and/or eliminated. The example in FIG. 5 may be implemented using any suitable components of computer system 270, such as interface 271 to interface with detector 231 and processor(s) 272 to generate phase-contrast and/or dark-field image data.

### (a) Planning Image Data (With Patient)

At 510 in FIG. 5, computer system 270 may obtain planning image data 110 that is acquired or generated using grating-based imaging system 201 to image patient 220 during pre-treatment phase 101. Using the example in FIGS. 1-2, control system 260 may generate and send control signal(s) to grating-based imaging system 201 during the imaging process. Computer system 270 may interface with detector 231 to obtain planning image data 110, which may be denoted as {∈} to represent a set of multiple (N) planning images for n=1, . . . , N.

Each planning image (¿) may be generated using imaging source 230 to emit imaging beam 250 towards multiple gratings 240-242 and detector 231. Patient 220 may be positioned between a pair of gratings, such as G0 240 and G1 241. Using phase stepping (explained using FIG. 3), each planning image (¿) may be associated with a particular phase step or grating position (pn) associated with G2 242, such as n(2π/N) using phase step size Δp=2π/N. Each planning image (¿) may represent intensity measurement data associated with multiple pixels of pixelated detector 231. A particular pixel may be denoted as (x, y).

### (b) Reference Image Data (Without Patient)

At 520 in FIG. 5, computer system 270 may obtain reference image data that is generated using grating-based imaging system 201 without any patient along the beamline (i.e., no subject imaged). The reference image data may be denoted as {Rn} to represent a set of multiple (N) reference images for n=1, . . . , N. Each reference image (Rn) may include reference intensity measurement data associated with multiple pixels of pixelated detector 231. Using phase stepping, each reference image (Rn) may be associated with a particular phase step or grating position (pn) associated with G2 242, such as n(2π/N) using phase step size=2π/N.

### (c) Phase-Stepping Curves

At 530 in FIG. 5, computer system 270 may determine, for a particular pixel (x, y), phase-stepping curves 531-532 associated with respective {∈} and {Rn}. In general, a phase-stepping curve is a periodic function that may be approximated using a sine function, etc. Phase-stepping curve 531-532 may each represent the intensity oscillations associated with various grating positions for a particular pixel (x, y). In the case where the intensity oscillations are sine functions, a fast Fourier transform (FFT) may be performed for each pixel (x, y) for the intensity oscillations. Alternatively, a least-squares algorithm for fitting a sine function to the data may be used.

Based on |¿|, first phase-stepping curve 531 (see “Curve 1” in FIG. 5) associated with pixel (x, y) may be generated, such as by plotting measured intensity data against its associated phase step position pn. From first phase-stepping curve 531, computer system 270 may extract first parameter data that includes (O₁, A₁, φ₁), where O₁=first intensity offset data or mean intensity data, A₁=first amplitude data and φ₁=first phase data.

Based on {Rn}, second phase-stepping curve 532 (see “Curve 2” in FIG. 5) associated with pixel (x, y) may be generated, such as by plotting intensity data that is measured without patient 220 against its associated phase step position pn. From second phase-stepping curve 532, computer system 270 may extract second parameter data that includes (O₂, A₂, φ₂), where O₂=second intensity offset data or mean intensity data, A₂=second amplitude data and φ₂=second phase data.

### (d) Image Data Generation

At 540 in FIG. 5, computer system 270 may generate P1 340 for a particular pixel (x, y) based on intensity offset data=(O₁, O₂) extracted from phase-stepping curves 531-532 associated with that pixel. In particular, computer system 270 may estimate P1 (x, y)=O₁/O₂, which is a ratio between (a) the first intensity offset data (O₁) associated with first curve 531 (i.e., with patient) and (b) the second intensity offset data (O₂) associated with second curve 532 (i.e., without patient). Block 540 may be repeated for all pixels.

At 550 in FIG. 5, computer system 270 may generate P2 350 based on phase data=(φ₁, φ₂) extracted from phase-stepping curves 531-532 associated with pixel (x, y). In particular, computer system 270 may estimate P2 (x, y)=φ₁−φ₂=Δφ, which represents the phase difference or phase shift between (a) the first phase data (φ₁) associated with first curve 531 (i.e., with patient) and (b) the second phase data (φ₂) associated with second curve 532 (i.e., without patient). Δφ represents a measurement parameter for P2 350. Block 550 may be repeated for all pixels.

At 560 in FIG. 5, computer system 270 may generate P3 360 based on intensity data=(O₁, O₂) and amplitude data=(A₁, A₂) extracted from phase-stepping curves 531-532 associated with pixel (x, y). First, computer system 270 may estimate V₁=A₁/O₁, which is first visibility data based on A₁=first amplitude data and O₁=first intensity offset data from first curve 531 (i.e., with patient). Next, computer system 270 may estimate V₂=A₂/O₂, which is second visibility data based on A₂=second amplitude data and O₂=second intensity offset data from second curve 532 (i.e., without patient). This way, computer system 270 may estimate dark-field image data associated with pixel (x, y) using P3 (x, y)=V₁/V₂=(A₁*O₂)/(A₂*O₁). Block 560 may be repeated for all pixels.

In practice, P1 340 (i.e., traditional X-ray images) may reveal the internal structure of soft tissue based on absorption contrast. P2 350 (i.e., phase-contrast X-ray images) may provide additional information by revealing phase changes within boundaries of target structure 310. Phase-contrast imaging may offer greater imaging sensitivity compared to conventional absorption-based imaging, particularly for low-density or low-absorbing materials. P3 360 (i.e., dark-field X-ray images) may provide additional information by revealing structures that scatter X-rays, such as micro-structures within soft tissue, etc.

Although explained using FIG. 5, it should be understood that any additional and/or alternative techniques may be used for image data generation. For example, techniques for exploiting moiré patterns may be implemented to generate P2 350 and/or P3 360. In general, moiré patterns are interference patterns that may occur when two regular patterns (e.g., gratings) overlap and interfere with each other. The moiré patterns may be residual or introduced on purpose.

### (e) Motion Artifacts

According to examples of the present disclosure, motion artifacts may be exploited. Here, the term “motion artifact” may refer generally to image data degradation that is caused by patient motion during image acquisition. The motion may be voluntary or involuntary (e.g., respiration or cardiac motion). In practice, any movement (e.g., in the order of micrometers) of target structure 310 during one phase step, or between multiple phase steps, may result in motion artifacts at the border of target structure 310. These motion artifacts may be exploited to solve the task of motion detection more efficiently. Depending on the desired implementation, motion artifacts may also speed up the image acquisition process.

An example will be explained using FIG. 6, which is example diagram 600 illustrating example phase stepping curves 640-660 that are generated based on planning image data 610, 630 and reference image data 620. Here, first planning image data 610 may be a set of first planning images that are denoted as {∈} for n=1, . . . , N and respective grating positions. Similar to the example in FIG. 5, |¿| may be generated using grating-based imaging system 201 to image target structure 310 within patient 220 who is positioned in a beam line associated with imaging beam 250 (see FIG. 3). Based on |¿|, first phase stepping curve 640 (see “Curve 1”) may be generated.

Reference image data 620 may be a set of reference images that are denoted as {Rn} for n=1, . . . , N and respective grating positions (pn). Similar to the example in FIG. 5, {Rn} may be generated using grating-based imaging system 201 without patient 220 in a beam line associated with imaging beam 250 (see FIG. 3). Based on {Rn}, second phase stepping curve 650 (see “Curve 2”) may be generated.

Second planning image data 630 may be a set of second planning images that are denoted as {Jn} and generated using grating-based imaging system 201 with patient 220 in the beam line associated with imaging beam 250 (see FIG. 3). Based on {Jn}, third phase stepping curve 660 (see “Curve 3”) may be generated. Compared with |¿|, patient 220 may have moved during the imaging process, thereby introducing motion artifact(s) into {Jn}. For example, target structure 310 of patient 220 may move out of the beam line between grating positions 9 and 10 (see 670 in FIG. 6).

For a first case (i.e., substantially low motion or no motion), image data generation may be performed based on parameter data extracted from “Curve 1” 640 and reference “Curve 2” 650, such as P1=0.6, P2=0.4 and P3=1.33 for a particular pixel (x, y). For a second case (i.e., with motion), image data generation may be performed based on parameter data extracted from “Curve 3” 660 and reference “Curve 2”650, such as P1′=0.65, P2′=0.89 and P3′=1.57 for a particular pixel (x, y). Comparing these values to neighboring pixel (x′, y′) associated with air only (to get the contrast), (ΔP1=0.4, ΔP2=0.4, ΔP3=0.33) for the first case and (ΔP1′=0.35, ΔP2′=0.89, ΔP3′=0.57) for the second case.

Based on the example in FIG. 6, it may be observed that motion artifacts introduced by patient 220 may result in an increased contrast compared to air at the boundary in the second case when compared to the first case. Such change in signal (or contrast) compared to a prior measurement in a series of measurements may be exploited to detect patient movement. This is similar to the classical attenuation-based “digital subtraction imaging”technique.

### Derived Image Data

Block 430 in FIG. 4 will be explained further using FIG. 7, which is a schematic diagram illustrating example process 700 for processing image data and generating derived image data during pre-treatment phase 101.

### (a) Metric-Based Selection

At 710-730 in FIG. 7, computer system 270 may implement a metric-based approach to select at least P2 350 and/or P3 360 for subsequent target structure tracking. For a particular Pj, computer system 270 may determine metric data (Mj) associated with Pj. Any suitable metric data may be determined, such as contrast, contrast to noise ratio, etc. In response to determination that Mj satisfies a particular threshold (e.g., first threshold for contrast or second threshold for contrast-to-noise ratio exceeded), associated Pj may be selected. Note that j [1, 2, 3] representing P1 340 (j=1), P2 350 (j=2) and P3 360 (j=3). This way, selected Pj may be used for template generation at block 750.

In one example, in response to determination that first metric data (e.g., M2 for j=2) associated with P2 350 satisfies a first threshold, P2 350 may be selected for use in subsequent template generation. Additionally or alternatively, in response to determination that second metric data (e.g., M3 for j=3) associated with P3 360 satisfies the first threshold or a second threshold, P3 360 may be selected for use in subsequent template generation.

### (b) Derived Image Data

Additionally or alternatively, at 740 in FIG. 7, computer system 270 may generate P4=derived image data based on at least one of P1 340, P2 350 and P3 360 according to blocks 741-746. At 741-742 in FIG. 7, computer system 270 may generate P4 by applying a function on one type of image data. For example, first function=f1(P2 ) may be applied to process P2 350 only. In another example, second function=f2(P3 ) may be applied to process P3 360 only. Blocks 741 and/or 742 may be performed to derive an absolute value for each pixel in image data 350/360, or threshold the value according to any suitable approach.

At 743-746 in FIG. 7, computer system 270 may generate P4 by applying a function on at least two of P1 340, P2 350 and P3 360. One or more of the following may be used: P4=f3(P1, P2, P3), P4=f4(P1, P2), P4=f5(P1, P3) and P4=f6(P2, P3). Any suitable function may be applied, such as linear combination function, non-linear combination function, ratio function, etc. A first linear combination function to combine or fuse various image data may be in the form of P4=a*P1+b*P2+c*P3 using coefficients (a, b, c). Another second example linear combination function may be in the form of P4=f4(P2, P3)=b*P2+c*P3 . In another example, P4=f3(P1, P3)=P3/P1 may be determined. Since P1 is a measure of absorption and P3 is a measure of scattering power, P4 may be described as scattering power per absorption. This measure may be used to facilitate the differentiation between different types of tissues.

In one example, P4=f5(P2 ) may be used as an input segmentation mask to process other image data (e.g., P1 340, P2 350 or P4) selective on the pixel-wise values of P2 350. In this case, since P2 350 is a differential image, it may be used as a segmentation mask to identify the boundaries of target structure 310. The segmentation mask may be used in any process (e.g., template generation) that requires information on the edges (e.g., edge enhancement algorithm) to process only certain image areas.

### Example Template Generation

At 750 in FIG. 7, computer system 270 may perform template generation by processing input data that includes at least one of P2 350 (i.e., phase-contrast image data), P3 360 (i.e., dark-field image data) and or any derivation thereof (e.g., P4). Any additional information may also be used to generate template image data, such as information relating to contoured structures (e.g., drawn by physician or determined using software/AI engine(s) for segmentation; see volume image data 132 in FIG. 1), isocenter of a treatment plan, etc.

In practice, template image data generated according to examples of the present disclosure may have improved characteristics, such as soft tissue contrast and/or target structure visibility. Such improvement may in lead to better target structure tracking and treatment outcomes, where target dose coverage may be effectively maintained while shrinking margins and increasing safety. Any suitable template generation approach may be implemented. Two examples will be discussed below.

### (a) Set of K Template Images

FIG. 8A is a schematic diagram illustrating first example process 800 for template generation based on phase-contrast and/or dark-field image data. In the example in FIG. 8A, a set of K template images (see 840-843) associated with respective K gantry angles may be generated. For a particular gantry angle □ₖ, where k∈[0, . . . , K−1], template generation may involve obtaining input data (see 810-812) that includes at least one P2 350, P3 360 and P4, identifying region(s) of interest (ROI), and extracting the ROI from the input data.

Some examples are shown in FIG. 8A using K=360 gantry angles spaced at L=1 degree. For k=0, a first template image (denoted as Tₖ₌₀) associated with a first gantry angle (□₀=0 degree) may be generated by extracting a first ROI (ROIₖ₌₀) from first input data associated with the same gantry angle; see 810, 820, 830 and 840. For k=1, second template image (denoted as Tₖ₌₁) associated with a second gantry angle □₁=1° may be generated by extracting a second ROI (ROIₖ₌₁) from input data associated with □₁; see 811, 821, 831 and 841. The same approach may be repeated for other gantry angles (□ₖ) until K=360 template images are generated, including □_{K-1} (see 812, 822, 832 and 842). Each template image (Tₖ) may be associated with at least one part of target structure 310 requiring radiation therapy.

Depending on the desired implementation, any suitable region or regions of interest may be defined for some or all gantry angles. As used herein, the term “region of interest” may refer generally to any suitable structure(s) extractable from input data, such as target structure (e.g., tumor), OAR (e.g., heart, spinal cord), bony landmark (e.g., vertebrae), soft tissue structures, air cavities, or any combination thereof, etc. In practice, ROI may be identified based on segmentation data, such as volume image data 132 (see block 131 in FIG. 1). In another example, ROI may be in the form of a volume of interest (VOI) that includes voxels in at least a subset of volume image data 132. Any additional and/or alternative approach for template generation may be implemented. One example may be found in U.S. Pat. No. 9,008,398 entitled “Template matching method for image-based detection and tracking of irregular shaped targets,”which is incorporated herein by reference.

### (b) AI-Based Approach

FIG. 8B is a schematic diagram illustrating second example process 801 for template generation based on phase-contrast and/or dark-field image data using an AI engine. As used herein, the term “AI engine” may refer to any suitable hardware and/or software component(s) of a computer system that are capable of executing algorithms according to any suitable AI model(s). Depending on the desired implementation, an “AI engine” may be a machine learning engine based on machine learning model(s), deep learning engine based on deep learning model(s), etc. In general, deep learning is a subset of machine learning in which multi-layered neural networks may be used for feature extraction as well as pattern analysis and/or classification.

Depending on the desired implementation, any suitable AI model(s) may be used, such as convolutional neural network, recurrent neural network, deep belief network, generative adversarial network (GAN), autoencoder(s), variational autoencoder(s), long short-term memory architecture for tracking purposes, generative AI model, transformer network, or any combination thereof, etc. In practice, a neural network is generally formed using a network of processing elements (called “neurons,” “nodes,” etc.) that are interconnected via connections (called “synapses,” “weight data,” etc.). A processing layer of a convolutional neural network may be a convolutional layer, pooling layer, un-pooling layer, rectified linear units (ReLU) layer, fully connected layer, loss layer, activation layer, dropout layer, transpose convolutional layer, concatenation layer, attention layer, or any combination thereof, etc. For example, convolutional neural networks may be implemented using any suitable architecture(s), such as UNet, LeNet, AlexNet, ResNet, VNet, DenseNet, OctNet, etc.

In the example in FIG. 8B, AI engine 850 may be implemented to process and map (a) input data 840=image data associated with one or more gantry angles to (b) output data 860=template image data associated with the gantry angle(s). Similar to the example in FIG. 8A, input data 840 may include {Pj}, where jϵ[1, 2, 3], selected at block 730 in FIG. 7 and/or P4 derived at block 740 in FIG. 7. AI engine 850 may include a hierarchy of multiple (X) processing layers (denoted as A₁ to A_{X}), such as an input layer, an output layer, and multiple (i.e., two or more) “hidden” layers between the input and output layers. The processing layers (A₁ to A_{X}) are associated with respective weight data (w₁ to w_{X}). During training, AI engine 850 may learn weight data (w₁ to w_{X}) to perform template generation based on input data 840 to generate template image.

AI engine 850 may be trained using any suitable approach, such as supervised learning, unsupervised learning, semi-supervised learning, reinforcement learning, etc. For example, in supervised learning, AI engine 850 may be trained on a dataset of labeled examples in order to learn the relationship between (a) input data=image data (e.g., phase-contrast/dark-field/derived image data, or any combination thereof) showing at least part of a target structure and (b) output data=template image data associated with the target structure. Any suitable training data may be used, such as synthetic data such as digitally reconstructed radiographs (DRRs), real patient data, or a combination of both. AI engine 850 may be trained using training data that is specific to patient 220, or a large variation of possible patients. In practice, a patient-specific training strategy may tackle the issue of inter-patient and inter-tumor variations (e.g., tumor size, shape, location, motion). In this case, DRRs may be synthetically generated for every degree of a full gantry arc (360°).

Alternatively, in unsupervised learning, AI engine 850 may be trained on a dataset of unlabeled examples in order to learn patterns and relationships in the data without any prior knowledge of the output labels. In semi-supervised learning, both labeled and unlabeled data may be used. Semi-supervised learning is useful in situations where there is a large amount of unlabeled data available, but it might be too expensive or difficult to label all of it. In reinforcement learning, AI engine 850 may learn to perform template generation by trial and error where it is rewarded for taking actions that lead to desired outcomes and penalized for taking actions that lead to undesired outcomes.

Depending on the desired implementation, P2 350, P3 360 and/or P4 may be used as training data to train a motion model (e.g., AI engine; not shown) for target structure tracking during treatment phase 102. Such a motion model may be used to model motion, such as inhalation and exhalation based on finite element method (FEM) modelling, etc. The training data may include a time series of projections from one angle together with data from a set of 3D scans and any suitable physics model(s) to train the motion model to provide predicted motion data.

### Example Template-Based Target Structure Tracking

According to examples of the present disclosure, target structure tracking may be performed during treatment phase 102 based on template image data that is generated during pre-treatment phase 101. An example use case will be explained using FIG. 9, which is a flowchart of example process 900 for a computer system to perform target structure tracking based on template image data. Example process 900 may include one or more operations, functions, or actions illustrated by one or more blocks, such as 910 to 950. Depending on the desired implementation, various blocks may be combined into fewer blocks, divided into additional blocks, and/or eliminated.

FIG. 9 will be described using FIG. 10, which is example radiation therapy system 1000 that includes a grating-based imaging system 1040 capable of performing phase-contrast and/or dark-field imaging during treatment phase 102. Using the example in FIG. 10, example process 900 may be performed using computer system 1060 capable of acting as a target structure tracking system. Computer system 1060 may include any suitable module(s) or component(s) such as interface 1061 to interface with radiation therapy system 1000 to perform block 910, image data processor(s) 1062 to perform blocks 920-925, tracking processor(s) 1063 to perform blocks 930-940, etc.

In practice, computer system 1060 may be located in the same physical location as radiation therapy system 1000, or in a different location. In both cases, computer system 1060 may be communicatively coupled with radiation therapy system 1000 via any suitable communication network(s). Computer system 1060 may be implemented using one or more physical machines (bare metal machines) and/or virtual machines deployed in a cloud-based environment. Control system 1050 and computer system 1060 may include any display device(s) and user input device(s), which are not shown for simplicity.

### (a) Treatment Image Data

At 910 in FIG. 9, computer system 1060 may obtain treatment image data 140 that is generated using an imaging source to emit an imaging beam towards a patient and a detector to image a target structure within the patient during treatment phase 102. In one example, treatment image data 140 may be acquired using a radiation therapy system without a grating interferometer. In this case, treatment image data 140 (i.e., absorption image data) may be used during subsequent target structure tracking in block 940.

Alternatively, at 911 in FIG. 9, treatment image data 140 may be acquired using radiation therapy system 1000 that includes grating-based imaging system 1040 in FIG. 10. Depending on the desired implementation, at 912 in FIG. 9, patient 220 may be administered with targeted contrast agents or biological tracers to enhance target visibility and improve the detectability of specific cells or cell clusters. Examples discussed with reference to block 411 in FIG. 4 are also applicable here and not repeated for brevity.

In the example in FIG. 10, radiation therapy system 100 may be configured to facilitate kilovolt (kV) imaging using kV imaging beam (see 1043) during application of a megavolt (MV) treatment beam (see 1030). Treatment image data 140 in the form of kV projection image data may be subsequently processed to generate phase-contrast and/or dark-field image data for target structure tracking according to examples of the present disclosure. One example treatment technique may be VMAT, where gantry 1010 is rotated around patient 220 during radiation therapy. Another example treatment technique may be static IMRT that is delivered with multi-leaf collimator (MLC). In a further example, proton treatment machine that delivers treatment using protons instead of X-ray radiation may be used. In practice, treatment image data 140 may include two dimensional (2D) projection image data, such as single energy (SE) or dual energy (DE) CBCT projection image data, etc.

To facilitate treatment delivery, radiation therapy system 1000 may include a radiation source in the form of linear accelerator (LINAC) 1021 as well as an imager/detector in the form of MV electronic portal imaging device (EPID) 1022. LINAC 1021 may generate and direct MV treatment beam 1030 towards isocenter 1014 through a PTV while gantry 1010 rotates through a treatment arc. In practice, MV treatment beam 1030 may be within a high-energy range, such as 10 mega-electron volts or greater. Gantry 1010 may rotate about bore or opening 1011 when actuated by drive system 1015, which is controlled using control system 1050 and/or computer system 1060. Patient 220 may be placed on treatment couch 1012 during treatment.

In the example in FIG. 10, on-board kV imaging system 1040 may be a grating-based imaging system that is capable of performing phase-contrast and/or dark-field imaging. For example, grating-based imaging system 1040 may include imaging source 1041 (labelled “S”), detector 1042 and multiple gratings (labelled “G0” to “G2”) that are interposed between them. In the case of three gratings, first grating=source grating (G0) 1044, second grating=phase grating (G1) 1045 and third grating=analyzer grating (G2) 1046 may be positioned between source 1041 and detector 1042.

Grating-based imaging system 1040 may be mounted orthogonally to LINAC 121 while sharing the same isocenter 1014. Compared to LINAC 1021, kV imaging source 1041 may be capable of producing imaging or diagnostic energy in the range of kV, such as below 1060 kV, etc. In response to detecting imaging X-ray beams 1043 generated by imaging source 1041, detector 1042 (e.g., pixelated detector, flat-panel imager) may generate suitable projection image data 1080. Various descriptions relating to gratings 240-242 in FIG. 2 are also applicable to gratings 1044-1046 in FIG. 10 and not repeated here for brevity.

### (b) Processing Treatment Image Data

At 920 in FIG. 9, computer system 1060 may process treatment image data 140 acquired using grating-based imaging system 1040 to generate one or more of the following: absorption treatment image data (P1*) 161, phase-contrast treatment image data (P2*) 162, dark-field treatment image data (P3*) 163. Note that the term “absorption/phase-contrast/dark-field treatment image data” may refer generally to absorption/phase-contrast/dark-field image data that is generated based on treatment image data 140 acquired during treatment phase 102, rather than planning image data 110 acquired during pre-treatment phase 101.

Depending on the desired implementation, at 925 in FIG. 9, computer system 1060 may generate derived treatment image data (P4*) 164 based on P2* 162 and/or P3* 163. Various descriptions relating to derived image data in FIG. 7 are also applicable here and not repeated for brevity. In practice, P2* 162, P3* 163 and P4* 164 may have improved characteristics compared to P1* 161, such as better target visibility and soft tissue contrast to facilitate target structure tracking. See 161-164 in FIG. 10.

### (c) Template Selection

At 940-941 in FIG. 9, computer system 1060 may select template image data that is generated based on at least one of the following: (a) P2 350, (b) P3 360 and (c) derived image data (P4) that is generated based on P2 350 or P3 360. The template image data (i.e., one or more template images) may be selected from a set of K template images associated with respective K gantry angles (θ₀, . . . , θ_{K-1}). For example, the selected template image data may be associated with a gantry angle (θₖ) that is closest to an imaging angle associated with treatment image data 140. The selected template image data may include one or more template images. Various descriptions relating to template image data in FIGS. 8A-B are also applicable here and not repeated for brevity.

### (d) Template Matching

At 950 in FIG. 9, computer system 1060 may perform target structure tracking based on the selected template image data and treatment image data 140. Block 950 may involve determining position data associated with the target structure by performing template matching. In one example (i.e., no grating-based imaging system), template matching may be performed based on absorption treatment image data 161/140. In another example, template matching may be performed based on at least one of the following: P2* 162, P3* 163 and P4* 164. See 951-952 in FIG. 9.

Depending on the desired implementation, template matching at block 950 may involve calculating a normalized cross correlation within a specific search region around an isocenter, which results in a match score value between 0 and 1. Calculating the match score at different pixel offsets produces a match score surface defined over the search region. A possible match may be indicated by the highest peak in the match score surface. However, in practice, this may be an incorrect match (especially for noisy images with little contrast). For each match, a peak-to-sidelobe-ratio (PSR) may also be calculated, where the PSR is the peak value divided by the standard deviation of the sidelobes. A minimum threshold for the match score value and PSR may be used to reject likely false matches.

In practice, template matching may be performed based on one selected template image (i.e., single template matching) or multiple selected template images (i.e., multi-template matching). The result of template matching may include 2D position data associated with the relevant target structure. Additionally, 3D position data may be estimated by performing triangulation based on the current 2D position data as well as the 2D position data associated with previous gantry angles.

### (e) Online Adjustment

Next, computer system 1060 may determine whether adjustment(s) are needed by comparing (a) the estimated position data calculated at block 950 with a (b) planned treatment position data based on which a treatment plan is generated during pre-treatment phase 101. In response to determination that the difference/deviation exceeds a predetermined threshold, computer system 1060 may may generate an alert that an adjustment is required. A deviation that exceeds the predetermined threshold may indicate that a significant portion of target structure 1023 is extending outside of a threshold region.

Based on the deviation detected, computer system 1060 may determine or estimate adjustment(s) to the patient setup (e.g., position or orientation of couch 1012) and/or treatment beam 1030 (e.g., gantry angle, collimator setup). Alternatively or additionally, instructions may be provided to patient 220 about the depth of breathing or depth of a breath hold in order to achieve the best match between treatment geometry (i.e., current 3D position data) and the planned geometry (i.e., planned position data).

Where applicable, treatment may be aborted. Using examples of the present disclosure, positional verification and target structure may be performed during radiation treatment in an improved manner to identify patients who move more than a predetermined threshold. This in turn enables adjustment(s) during treatment delivery phase 102 to achieve better treatment outcomes for patient 220.

In practice, motion management during radiation therapy remains an open problem. In some cases, conventional motion management techniques may contribute to large target margins, limiting the ability of clinicians to spare the healthy tissue surrounding the target structure. Regardless of the strategy used to adapt delivered radiation fields to match or otherwise manage motion of the target structure, the quality, latency, and/or information content within acquired real-time image data still needs improvement to reduce target margins.

### Computer System

The above examples can be implemented by hardware (including hardware logic circuitry), software or firmware or a combination thereof. The above examples may be implemented by any suitable computing device, computer system, etc. The computer system may include processor(s), memory unit(s) and physical NIC(s) that may communicate with each other via a communication bus, etc. Examples of the present disclosure may also include a non-transitory computer-readable storage medium that includes a set of instructions which, in response to execution by a processor of the computer system, cause the processor to perform target structure tracking described herein with reference to the drawings.

The techniques introduced above can be implemented in special-purpose hardwired circuitry, in software and/or firmware in conjunction with programmable circuitry, or in a combination thereof. Special-purpose hardwired circuitry may be in the form of, for example, one or more application-specific integrated circuits (ASICs), programmable logic devices (PLDs), field-programmable gate arrays (FPGAs), and others. The term ‘processor’ is to be interpreted broadly to include a processing unit, ASIC, logic unit, or programmable gate array etc.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or any combination thereof.

Those skilled in the art will recognize that some aspects of the embodiments disclosed herein, in whole or in part, can be equivalently implemented in integrated circuits, as one or more computer programs running on one or more computers (e.g., as one or more programs running on one or more computing systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure.

Software to implement the techniques introduced here may be stored on a non-transitory computer-readable storage medium and may be executed by one or more general-purpose or special-purpose programmable microprocessors. A “computer-readable storage medium”, as the term is used herein, includes any mechanism that provides (i.e., stores and/or transmits) information in a form accessible by a machine (e.g., a computer, network device, personal digital assistant (PDA), mobile device, manufacturing tool, any device with a set of one or more processors, etc.). A computer-readable storage medium may include recordable/non recordable media (e.g., read-only memory (ROM), random access memory (RAM), magnetic disk or optical storage media, flash memory devices, etc.).

The drawings are only illustrations of an example, wherein the units or procedure shown in the drawings are not necessarily essential for implementing the present disclosure. Those skilled in the art will understand that the units in the device in the examples can be arranged in the device in the examples as described or can be alternatively located in one or more devices different from that in the examples. The units in the examples described can be combined into one module or further divided into a plurality of sub-units.

1. A method for a computer system to perform image data processing for radiation therapy, wherein the method comprises:
obtaining planning image data that is generated using an imaging source to emit an imaging beam towards a patient and a detector to image a target structure within the patient during a pre-treatment phase of radiation therapy;
based on the planning image data, generating at least one of (a) phase-contrast image data associated with the target structure and (b) dark-field image data associated with the target structure; and
generating template image data associated with the target structure by processing at least one of the following: (a) the phase-contrast image data, (b) the dark-field image data and (c) derived image data that is generated based on the phase-contrast image data or the dark-field image data, wherein the template image data is generated for tracking the target structure during a treatment phase of the radiation therapy.

2. The method of claim 1, wherein obtaining the planning image data comprises:
obtaining the planning image data that is generated using a grating-based imaging system that includes the imaging source, the detector and multiple gratings that are positioned between the imaging source and the detector.

3. The method of claim 2, wherein generating at least one of (a) the phase-contrast image data and (b) the dark-field image data comprises:
determining first parameter data associated with the planning image data that includes a set of multiple planning images associated with a set of respective multiple phase steps, wherein the first parameter data includes first intensity offset data, first amplitude data and first phase data;
determining second parameter data associated with reference image data that is generated using the grating-based imaging system without the patient, wherein the reference image data includes a set of multiple reference images associated with the set of respective multiple phase steps, wherein the second parameter data includes second intensity offset data, second amplitude data and second phase data; and
based on the first parameter data and the second parameter data, generating (a) the phase-contrast image data, (b) the dark-field image data and (c) absorption image data.

4. The method of claim 1, wherein generating the template image data comprises at least one of the following:
in response to determination that first metric data associated with the phase-contrast image data satisfies a first threshold, selecting the phase-contrast image data for use in generating the template image data; and
in response to determination that second metric data associated with the dark-field image data satisfies the first threshold or a second threshold, selecting the dark-field image data for use in generating the template image data.

5. The method of claim 1, wherein the method further comprises:
generating the derived image data by applying a function to combine or calculate a ratio between at least two of the following: (a) the phase-contrast image data, (b) the dark-field image data and (c) absorption image data.

6. The method of claim 1, wherein generating the template image data comprises:
identifying one or more regions of interest in input data associated with a particular gantry angle, wherein the input data includes at least one of (a) the phase-contrast image data, (b) the dark-field image data, and (c) the derived image data; and
extracting the one or more regions of interest from the input data to generate a template image associated with the particular gantry angle.

7. The method of claim 1, wherein generating the template image data comprises:
generating the template image data using an artificial intelligence (AI) engine to process at least one of the following: (a) the phase-contrast image data, (b) the dark-field image data and (c) the derived image data, wherein the AI engine includes multiple processing layers that are trained to perform template generation.

8. A radiation therapy system, comprising:
a grating-based imaging system that includes an imaging source, a detector and multiple gratings that are positioned between the imaging source and the detector; and
a computer system configured to:
obtain, from the grating-based imaging system, planning image data that is generated using the imaging source to emit an imaging beam towards the multiple gratings and the detector to image a target structure within a patient during a pre-treatment phase of radiation therapy;
based on the planning image data, generate at least one of (a) phase-contrast image data associated with the target structure and (b) dark-field image data associated with the target structure; and
generate template image data associated with the target structure by processing at least one of the following: (a) the phase-contrast image data, (b) the dark-field image data and (c) derived image data that is generated based on the phase-contrast image data or the dark-field image data, wherein the template image data is generated for tracking the target structure during a treatment phase of the radiation therapy.

9. The radiation therapy system of claim 8, wherein the computer system is configured to generate at least one of (a) the phase-contrast image data and (b) the dark-field image data by:
determining first parameter data associated with the planning image data that includes a set of multiple planning images associated with a set of respective multiple phase steps, wherein the first parameter data includes first intensity offset data, first amplitude data and first phase data;
determining second parameter data associated with reference image data that is generated using the grating-based imaging system without the patient, wherein the reference image data includes a set of multiple reference images associated with the set of respective multiple phase steps, wherein the second parameter data includes second intensity offset data, second amplitude data and second phase data; and
based on the first parameter data and the second parameter data, generating (a) the phase-contrast image data, the dark-field image data and (c) absorption image data.

10. The radiation therapy system of claim 8, wherein the computer system is configured to generate the template image data by:
in response to determination that first metric data associated with the phase-contrast image data satisfies a threshold, selecting the phase-contrast image data for use in generating the template image data.

11. The radiation therapy system of claim 8, wherein the computer system is configured to generate the template image data by:
in response to determination that second metric data associated with the dark-field image data satisfies a threshold, selecting the dark-field image data for use in generating the template image data.

12. The radiation therapy system of claim 8, wherein the computer system is further configured to:
generate the derived image data by applying a function to combine or calculate a ratio between at least two of the following: (a) the phase-contrast image data, (b) the dark-field image data and (c) absorption image data.

13. The radiation therapy system of claim 8, wherein the computer system is configured to generate the template image data by:
identifying one or more regions of interest in input data associated with a particular gantry angle, wherein the input data includes at least one of (a) the phase-contrast image data, (b) the dark-field image data and (c) the derived image data; and
extracting the one or more regions of interest from the input data to generate a template image associated with the particular gantry angle.

14. The radiation therapy system of claim 8, wherein the computer system is configured to generate the template image data by:
generating the template image data using an artificial intelligence (AI) engine to process at least one of the following: (a) the phase-contrast image data, (b) the dark-field image data and (c) the derived image data, wherein the AI engine includes multiple processing layers that are trained to perform template generation.

15. A method for a computer system to perform target structure tracking for radiation therapy, wherein the method comprises:
obtaining treatment image data that is generated using an imaging source to emit an imaging beam towards a patient and a detector to image a target structure within the patient during a treatment phase of radiation therapy;
selecting template image data that is generated based on at least one of the following: (a) phase-contrast image data associated with the target structure, (b) dark-field image data associated with the target structure and (c) derived image data that is generated based on the phase-contrast image data or the dark-field image data; and
based on the selected template image data and the treatment image data, determining position data associated with the target structure, thereby tracking the target structure during the treatment phase.

16. The method of claim 15, wherein selecting the template image data comprises:
selecting the template image data that is generated based on planning image data that is acquired during a pre-treatment phase of radiation therapy, wherein the planning image data is processed to generate at least one of the following: (a) the phase-contrast image data, (b) the dark-field image data or (c) the derived image data.

17. The method of claim 15, wherein determining the position data comprises:
performing template matching to match (a) the treatment image data, being absorption treatment image data, to (b) the selected template image data that includes one or more template images associated with the target structure.

18. The method of claim 15, wherein determining the position data comprises:
processing the treatment image data to generate at least one of the following: (a) phase-contrast treatment image data associated with the target structure, (b) dark-field treatment image data associated with the target structure, and (c) derived treatment image data that is generated based on the phase-contrast treatment image data or the dark-field treatment image data.

19. The method of claim 18, wherein determining the position data comprises:
performing template matching based on the selected template image data and at least one of the following: (a) the phase-contrast treatment image data, (b) the dark-field treatment image data and (c) the derived treatment image data.

20. The method of claim 18, wherein obtaining the treatment image data comprises:
obtaining the treatment image data that is generated using a grating-based imaging system that includes the imaging source, the detector and multiple gratings that are positioned between the imaging source and the detector.
